(19) United States
(12) Patent Application Publication
(10) Pub. No.: US 2018/0221192 A1
(43) Pub. Date: Aug. 9, 2018

(54) Shoulder and Arm Support

(71) Applicants: Scott Edwin Strasburger, Lincoln, NE (US); Curtis Bryan Carlson, Hickman, NE (US)

(72) Inventors: Scott Edwin Strasburger, Lincoln, NE (US); Curtis Bryan Carlson, Hickman, NE (US)

(21) Appl. No.: 15/944,897

(22) Filed: Apr. 4, 2018

Related U.S. Application Data
(63) Continuation-in-part of application No. 15/155,468, filed on May 16, 2016.

(51) Int. Cl. A61F 5/37; A61F 5/01

(52) U.S. Cl. CPC A61F 5/3738; A61F 5/012; A61F 5/0118

(57) ABSTRACT

A shoulder and arm support for supporting either the left or right shoulder and arm of a patient. The support includes a case, a removable support member, and a sling which houses a shoulder strap secured thereto and a body strap secured thereto. The case houses an interior compartment in which a resilient cushion and an inflatable bladder are positioned. The case includes an outer panel which is pivotally secured to the upper panel of the case and movable between first and second positions of abduction. The inflation and deflation of a pneumatic bladder cause the outer panel to be moved continuously. The removable support member has an interior compartment which houses a resilient cushion. The exterior surface of the outer panel allows for a sling, making this shoulder and arm support customizable to the patient, position of comfort, and the phase of the rehabilitation process.

### BACKGROUND OF THE INVENTION

### Field of the Invention

This invention relates to a support which treats and protects the shoulder and arm of a person for numerous orthopedic conditions, following surgery or injury to the area. More particularly, this invention relates to an adjustable shoulder and arm support that provides rehabilitation through continuous passive motion, stations of abduction, and static positioning. More specifically, this invention relates to a bilateral and universal shoulder and arm support which may be used to assist the patient throughout the recovery process by providing a combination of customizable treatment options that address the various phases of the recovery and rehabilitation process.

### Description of the Related Art

The customary method for supporting the arm and shoulder of a person following an injury or surgery is to provide the patient with a sling which supports the shoulder against the body in a static position. Another option is to use a static shoulder abduction pillow, otherwise referred to as an immobilizer, which holds the arm away from the body in a fixed position and degree of abduction. These devices are insufficient for various indications, or surgical repairs, specifically because they keep the shoulder in a fixed position and are not adjustable to meet the specific needs of the patient. Following a shoulder injury or surgery, the post-operative positioning and position of comfort are extremely variable from patient to patient and from indication to indication. Prior art devices are not adjustable to provide support to the arm and shoulder in various degrees of abduction. The fact that the support holds the arm in a fixed position, increases the likelihood that the patient will develop adhesive capsulitis, or frozen shoulder, a significant complication following a shoulder injury or surgery. The less opportunity the patient has for movement, the greater risk of adhesive capsulitis.

Further, the prior art devices are not able to create continuous passive motion of the shoulder from 20 degrees of abduction to 90 degrees of abduction.

### SUMMARY OF THE INVENTION

This summary is provided to introduce a selection of concepts in a simplified form that is further described below in the detailed description. This summary is not intended to identify key aspects or essential aspects of the claimed subject matter. Moreover, this summary is not intended for use as an aid in determining the scope of the claimed subject matter.

The shoulder and arm support of this invention include a main body, or case, that can be worn bilaterally. The case, made of fleece or loop fabric, is formed by an upstanding inner panel having a curved inner side so as to conform to the side of the patient, an upstanding first and second side panels which extend outwardly from the sides of the inner panel, a horizontally disposed bottom panel, and a flat vertical back panel which connects to a bottom upstanding inner panel allowing space for a removable support member. The bottom horizontal panel houses a port providing an opening for a power cord and hand-held controller cord. The first and second side panels, of the bottom upstanding inner side panel, house two strips of hook fabric of a hook-and-loop fastener system. Together these form a compartment which houses a resilient polyurethane cushion.

A removable support member, made of loop or fleece fabric, is comprised of an inner panel having a curved inner side so as to conform to the side of the patient, first and second side panels which extend outwardly from the sides of the inner panel, a horizontally disposed bottom panel, and an outer back panel. Together, these create a compartment, or case, which hosts a resilient polyurethane cushion, that conforms to the side of the patient. The removable support member attaches to the hook fabric of the flat vertical back panel as well as the hook fabric located on the bottom upstanding inner side panel of the case.

The case also includes a horizontally-disposed upper panel which is secured to the upper ends of the inner panel, the first side panel, and the second side panel. The case further includes an outer panel which has its upper-end pivotally secured to the outer end of the end of the upper panel whereby the outer panel is pivotally movable between first and second positions. A flexible bellow, made of polyester or spandex, is provided and secured to the outer end of the bottom panel, the outer ends of the first and second side panels, and to the outer panel by stitching and the like.

The inner panel, the first side panel and the second side panel together with the bottom panel define an interior compartment which is closed by the bellow and the outer panel which houses a resilient foam cushion.

Multiple inflatable bladders comprise the bladder system and are positioned inside the bellow. The bladders, made of thermoplastic polyurethane, are configured to be moved between a collapsed and inflated position. The bladder system, upon being moved between its collapsed position and the inflated position, causes the outer panel to be moved from its first position towards its second position of abduction and returns to its first position, continuously within a therapy session. First and second positions of abduction include 20°, 45°, 60°, 75°, and 90°. In another embodiment, abduction extends beyond 90°. Timed therapy sessions can be customized at 15, 30 or 45 minutes based on the phase of the rehabilitation process. In another embodiment, the time intervals include alternative time intervals.

The outer panel, when in its first position, is disposed at a 20° of abduction with respect to vertical. In an additional embodiment, the first position of abduction can be customized to the physician's preferred degree of abduction and could include the range of 20° to 90° of abduction and beyond. The outer panel, when in its second position, is disposed of in a plane of about 90° or more with respect to vertical. The bladder system may be inflated to move the outer panel from its first position to its second position or may be used to continuously inflate or deflate the bladder system so that the upper panel is continuously moved between its first and second positions to rehabilitate the patient's shoulder.

A pump, used to inflate the bladder system, is positioned inside the bellow. An air hose connects the pump to the bladder system, allowing air to travel from the pump to the bladder system and cause it to inflate. The bladder system automatically deflates upon the completion of the therapy session or can be manually deflated using the deflation valve located on the side panels of the main case. In an additional embodiment, the deflation valve could be located in a different location on the shoulder and arm support and could be referred to as a pressure release valve, relief valve or safety release valve. A hand-held controller cord runs from the pump out the horizontal bottom panel through a port and attaches to a hand-held controller. The hand-held controller, made of acrylonitrile butadiene styrene plastic, houses a push-button for power, push-buttons for selecting the customized degrees of abduction, and push-buttons for selecting the length of the therapy session. When not in use, the hand-held controller is housed in a controller pouch which is attached to the first and second side panels. A power cord, attached to the pump, also runs through the port and connects to a power source. In an additional embodiment, the hand-held controller could be wireless.

The outer panel houses a horizontal strip of hook fabric of the hook-and-loop fastener system. A sling used to reduce the weight-bearing load after injury, made of fleece or loop fabric and attaches to the hook fabric on the outer panel of the case. A length-adjustable sling strap is attached to the sling pocket through D-rings. The sling hosts a thumb loop and a detachable hand support which prevents migration and provides support for the hand, arm, and shoulder of the patient by securing the arm and shoulder to the exterior surface of the outer panel. The sling is bilateral and accommodates various arm sizes, making it universal. In an additional embodiment, the sling is made of mesh material. The detachable hand support can also be attached to the removable support member or the case.

In another embodiment the exterior surface of the outer panel has a plurality of arm supports secured thereto. The arm supports are adjustably secured to the exterior surface of the outer panel to accommodate different arm sizes and to enable the device to be used to support either the left shoulder and arm or the right shoulder and arm of the patient.

A length adjustable shoulder strap, which houses a shoulder pad, provides comfort and is secured to the first and second side panels of the case through circular clips by stitching or the like.

Alignment brand marks span across both the first and second side panels of the upstanding inner panel and of the removable support member's upstanding inner panel. These marks provide assembly guides to the patient when attaching the removable support member to the case of the shoulder and arm support.

A large D-ring is attached to the first and second side panels of the removable support member allowing a length adjustable support strap to enclose the waist and secure the shoulder and arm support to the patient. The length adjustable support strap houses a buckle.

The indications for this shoulder and arm support include rotator cuff repairs, Bankart lesion, superior labrum anterior-posterior (SLAP) repair, impingement syndrome shoulder, adhesive capsulitis, reverse total shoulder replacement, shoulder replacement, capsular plication, rotator cuff tendonitis, calcific rotator cuff tendonitis, capsular release, labrum repair, subacromial bursectomy and decompression, and open reduction internal fixation (ORIF) of a proximal humerus fracture, among others.

The shoulder and arm support disclosed can be used across the rehabilitation process by separating the components based on the patient's need. This 3-in-1 arm and shoulder support can be worn as an individual sling, as a sling attached to the removable support member creating an immobilizer, or as previously disclosed comprising: the case, removable support member, and the sling, attached and complete.

These and other objects will be apparent to those skilled in the art. The preferred embodiment of the materials used to make the shoulder and arm support are disclosed. This material is readily available, and those of skill in the art are familiar with working with such materials. Other materials are available that would be suitable for alternative embodiments of the subject matter of the disclosure. Examples are cotton or cotton blended fleece, polyester fleece, lycra spandex fleece, Microfleece or any other similar materials. Those in the art will understand that in any suitable material, now known or hereafter developed, may be used in forming the shoulder and arm support described herein.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a front perspective view illustrating the shoulder and arm support of this invention being secured to the left side of an individual;

FIG. 2 is a side view of the invention illustrating the shoulder and support being raised to a particular position;

FIG. 3 is a back view of the shoulder and arm support maintaining the arm and shoulder of the patient in a fixed elevated position;

FIG. 4 is a perspective view of the shoulder and arm support of this invention;

FIG. 5 is an exploded perspective view of the shoulder and arm support of this invention including the stiffener plate, contoured rigid plate, case and cushion, bladder system, pump and hand-held controller;

FIG. 6 is an exploded perspective view of the shoulder and arm support including the sling, case, shoulder straps, removable support member and waist strap with a portion thereof cut-away to more fully illustrate the invention;

FIG. 7 is a perspective view of the bladder system, pump and hand-held controller of this invention;

FIG. 8 is a front view of the shoulder and arm support in an elevated position of the patient may be raised and lowered for rehab purposes with portions thereof cut-away to more fully illustrate the invention; and

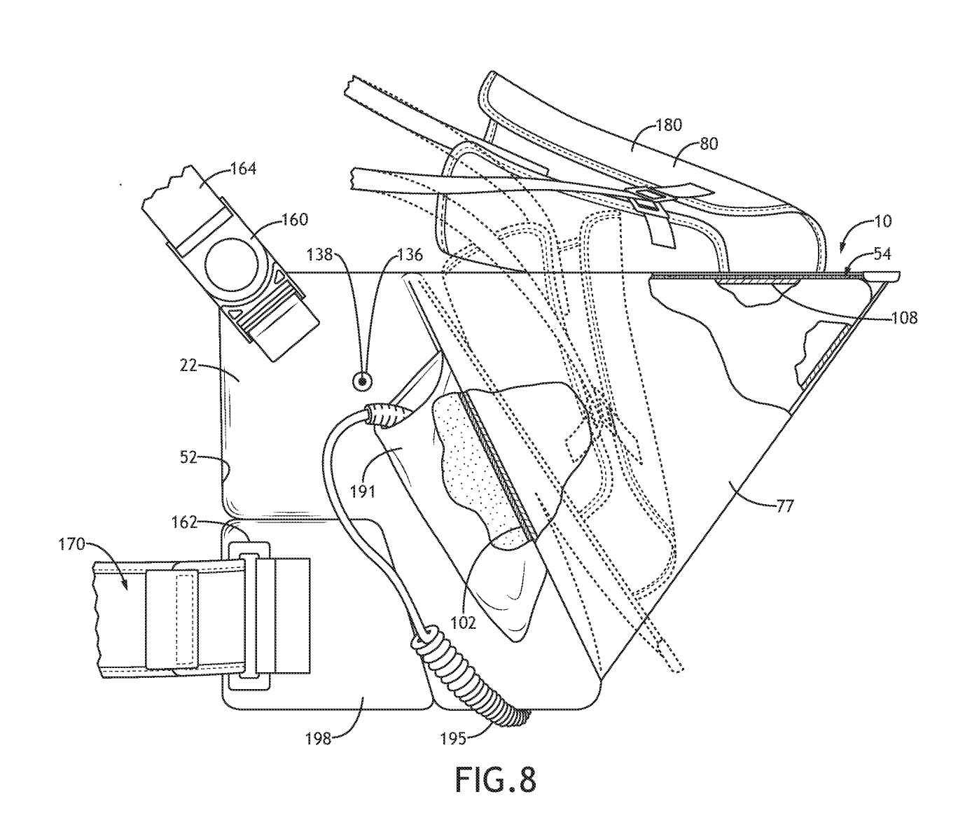

FIG. 9 is a posterior/bottom view of the case of the shoulder and arm support highlighting the port;

FIG. 10 is a side view of the case of the shoulder and arm support without the removable support member.

### DESCRIPTION OF THE PREFERRED EMBODIMENT

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense in that the scope of the present invention is defined only by the appended claims.

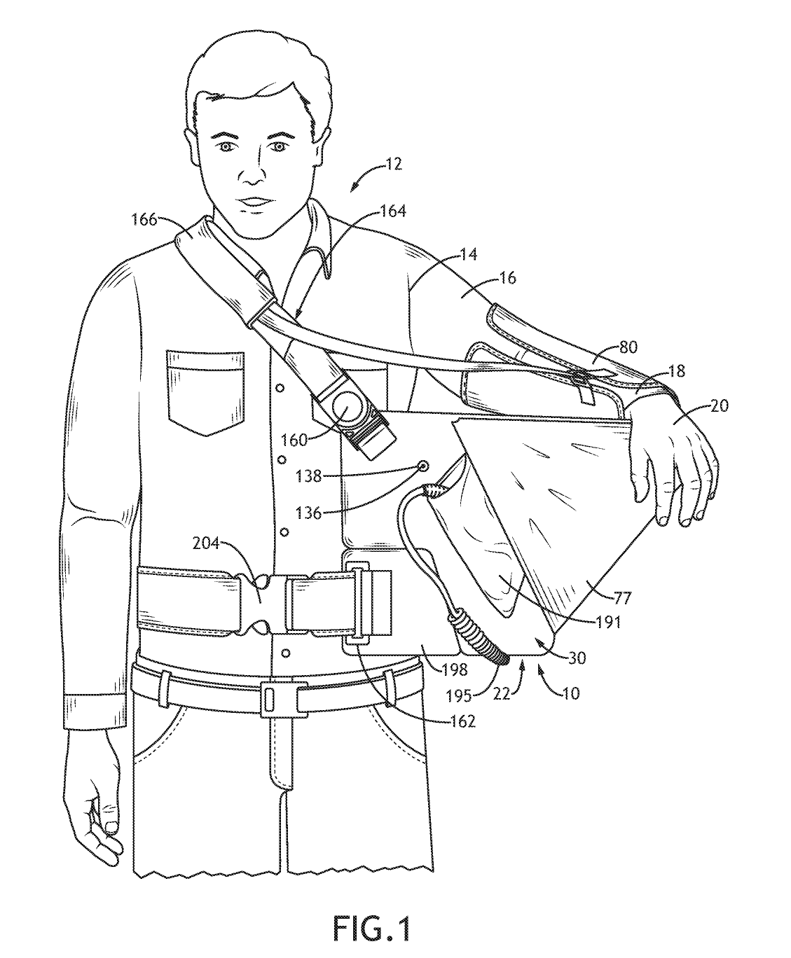

The numeral 10 refers to the shoulder and arm support of this invention which is designed to be used at either side of a person 12 having a shoulder 14, upper arm 16, lower arm 18 and hand 20 as seen in FIG. 1.

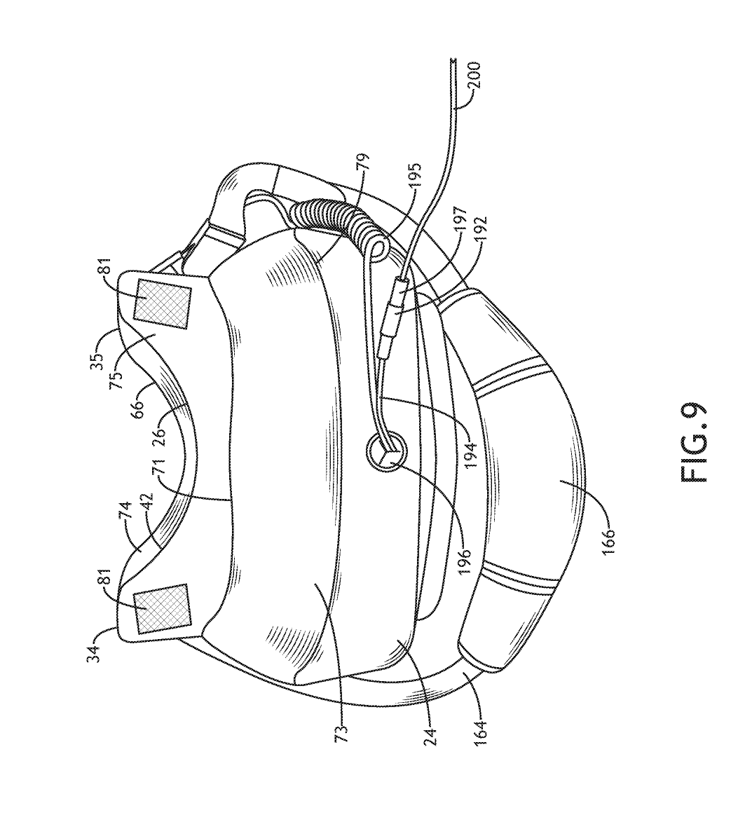

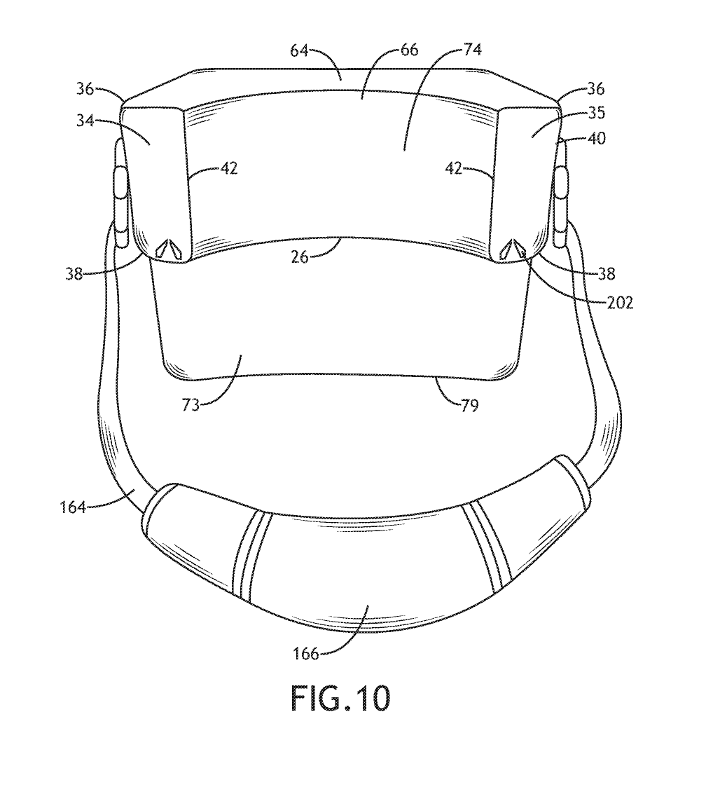

The shoulder and arm support 10 includes a case 22 which includes a horizontal bottom panel 24 having a curved inner bottom end 79, a first side panel 28, a second side panel 30, and a top panel 64. Case 22 also includes an upstanding first side panel 34, and an upstanding second side panel 35, both having an upper end 36, an outer end 40, a lower end 38 and an inner end 42, as seen in FIGS. 9 and 10.

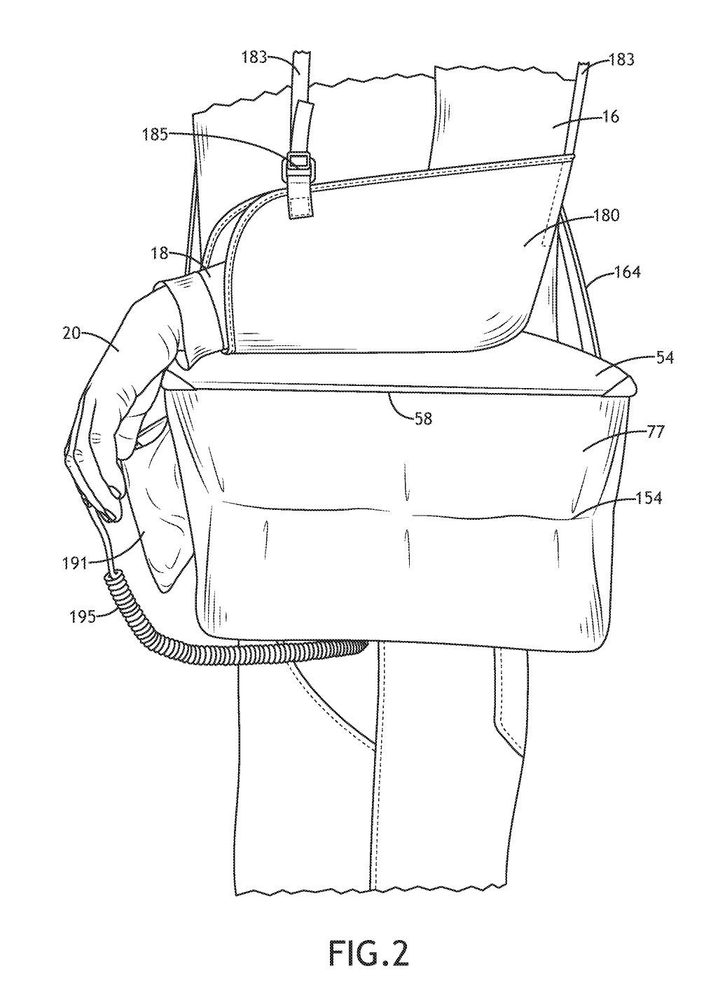

Case 22 further includes an outer panel 54, as seen in FIG. 2, having an upper end 56, a lower end 58, a first side outer end 60 and a second side outer end 62. First side outer end 60, of outer panel 54, is joined to the upper first side end 148 of, bellow 77, by stitching or the like. Second side outer end 62, of outer panel 54, is joined to upper second side end 152, of bellow 77, by stitching or the like. The lower end 58, of outer panel 54, is joined to the lower end 153, of bellow 77, by stitching or the like. The lower first side portion 146 and lower second side portion 150 is connected to the outer end 40 of the upstanding first and second side panels 28 and 30 respectively.

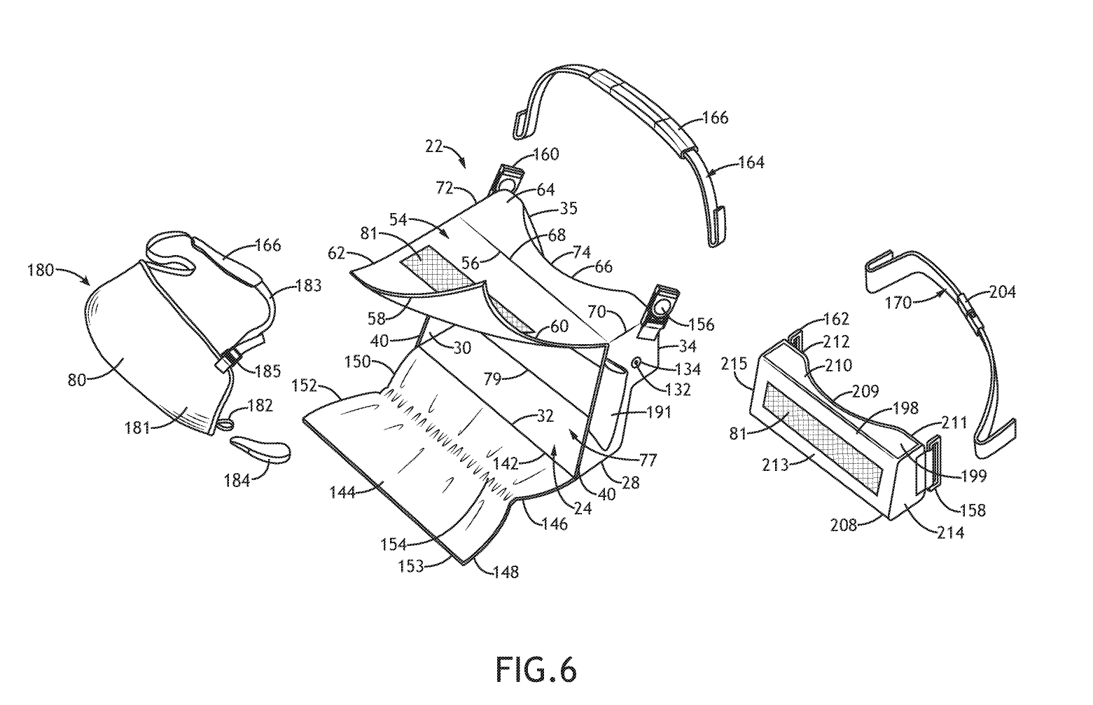

The top panel 64, of the case 22, has a curved inner end 66, an outer end 68, a first side top end 70, and a second side top end 72 as seen in FIG. 6. The outer end 68, of top panel 64, is joined to the upper end 56 of the outer panel 54, by stitching or the like. The first side top end 70, of top panel 64, is joined to the upper end 36, of the first side panel 28, by stitching or the like. The second side top end 72, of top panel 64, is joined to the upper end 36 of the second side panel 30, by stitching or the like.

The case 22, as seen in FIGS. 9 and 10, also includes an upstanding curved inner panel 74 which has its upper end secured to the curved inner end 66, of top panel 64, by stitching or the like, which has its lower end secured to the curved inner bottom end 26 of the bottom upstanding inner panel 75 by stitching or the like. The bottom upstanding inner panel 75 connects to a flat vertical back panel 73 allowing space for a removable support member 198 which also conforms to the patient. The bottom upstanding inner end 71 connects the bottom upstanding inner panel 75 to the flat vertical back panel 73 by stitching or the like. The flat vertical back panel 73 connects to the horizontal bottom panel 24 by the flat vertical back bottom end 79. The bottom upstanding inner panel 75 also houses hook fabric 81 of hook-and-loop fastener system on both the first and second side.

The bottom horizontal panel 24 also houses a port 196 which allows an opening for power cord 194 and a hand-held controller cord 195 to pass through, as seen in FIG. 9.

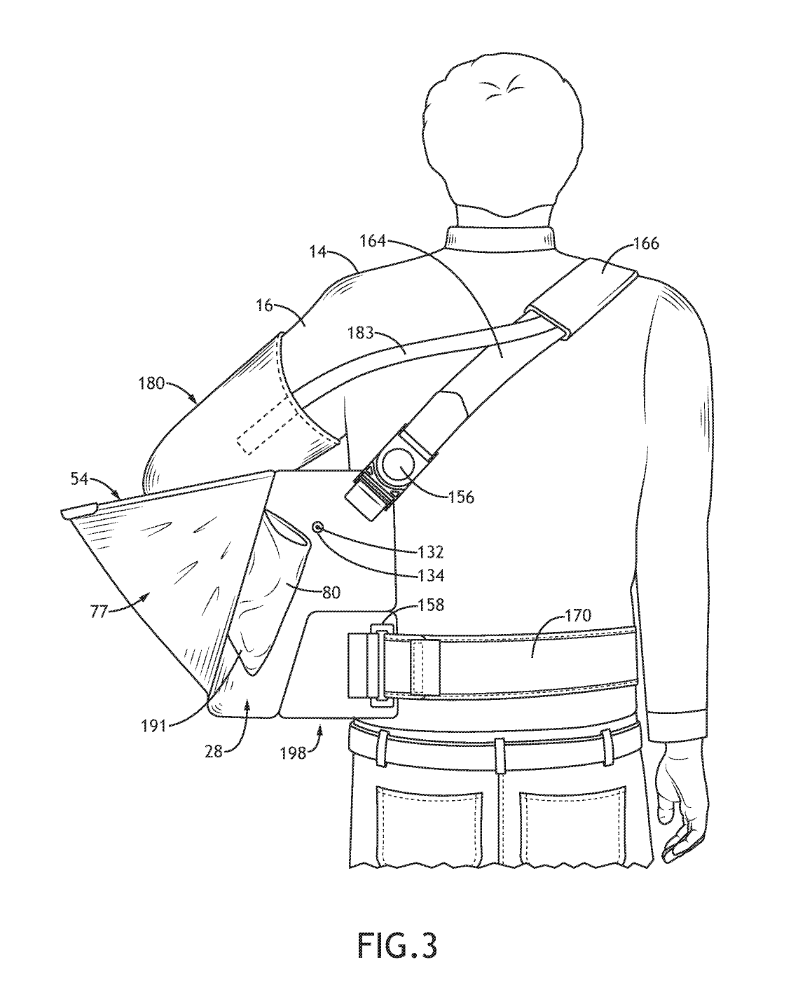

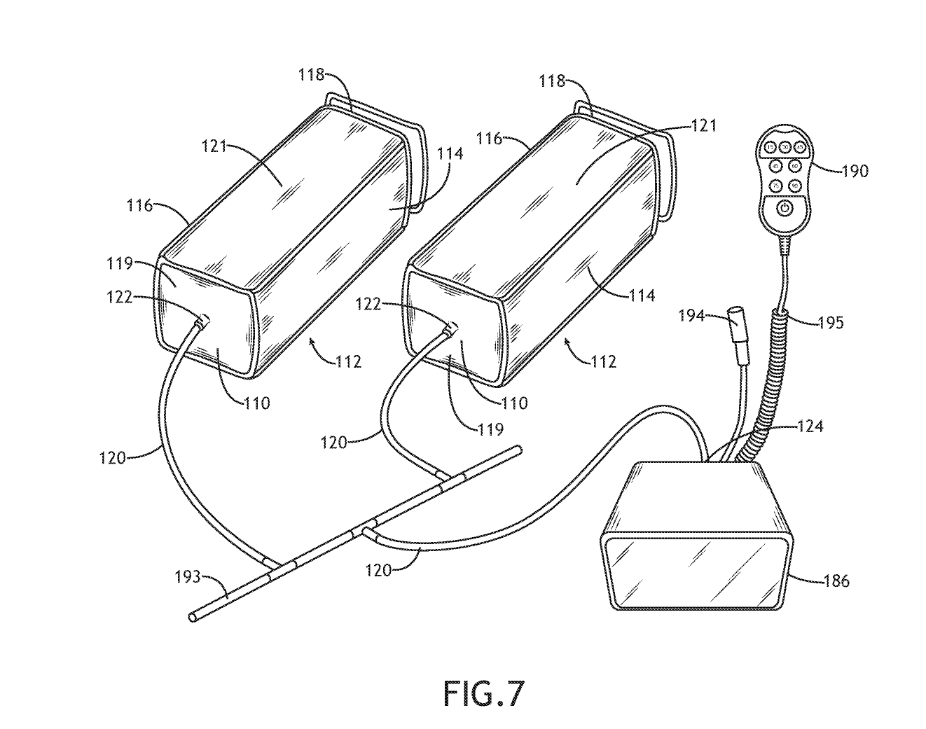

A deflation valve 132 is located on the first side panel 28 and a second deflation valve 136 is located on the second side panel 30, as seen in FIGS. 1 and 3, connecting to an air hose system 120 which runs through a T-connector 193 bilaterally and connects to the bladder system 110. Although the preferred embodiment depicted in FIG. 7 includes a t-connector connecting several separate pieces of tubing, referred to as the air hose system, those in the art will understand that the t-connector can be comprised of one length of tubing or of several pieces of tubing, all functioning as a t-connector and air hose system.

The bladder system 110, as seen in FIG. 7, consists of multiple bladders made of thermoplastic polyurethane and is moved from a collapsed position to a fully inflated position. The bladders of the bladder system 110 include a bottom panel(s) 112, an outer panel(s) 114, an inner panel(s) 116, a first side panel(s) 118, a second side panel(s) 119 and a top panel(s) 121. The air hose system 120 has its inner end(s) 122 in communication with the interior of bladder system 110 at side panel(s) 119 and extends therefrom to its outer end(s) 124 which connects to the pump 186. The air hose system 120 extends outwardly and communicates with the deflation valves 132 and 136 located on the first and second side panels 28 and 30 respectively of the case 22. The numeral 76 refers to a polyurethane foam cushion which is enclosed in case 22. Other materials are available that would be suitable for alternative embodiments of the polyurethane foam. Examples are latex foam, or any other similar materials. Those in the art will understand that in any suitable material, now known or hereafter developed, may be used in forming the shoulder and arm support described herein.

Although the preferred embodiment depicted in FIG. 7 includes two individual bladders, those in the art will understand that the bladder system can comprise one bladder or a group of several bladders, all functioning as a bladder system.

The bellow 77, as seen in FIG. 6, includes an inner end 142, an outer end 144, a lower first side portion 146, an upper first side portion 148, a lower second side portion 150 and upper second side portion 152. Bellow 77 includes at least one elastic band or strap 154 secured thereto by stitching or the like and extends thereacross. The inner end 142, of bellow 77, is secured to the outer end 32 of horizontal bottom panel 24 by stitching or the like. The lower first side portion 146 is secured to the outer end 40 of the first side panel 28. The upper first side portion 148 is secured to the first side outer panel 60 of the outer panel 54 by stitching or the like. The lower second side portion 150 is secured to the outer end 40 of the second side panel 30 by stitching or the like. The upper second side portion 152 is secured to the second side outer panel 62 of outer panel 54 by stitching or the like. The outer end 144 is secured to lower end outer panel 58 of outer panel 54 by stitching or the like.

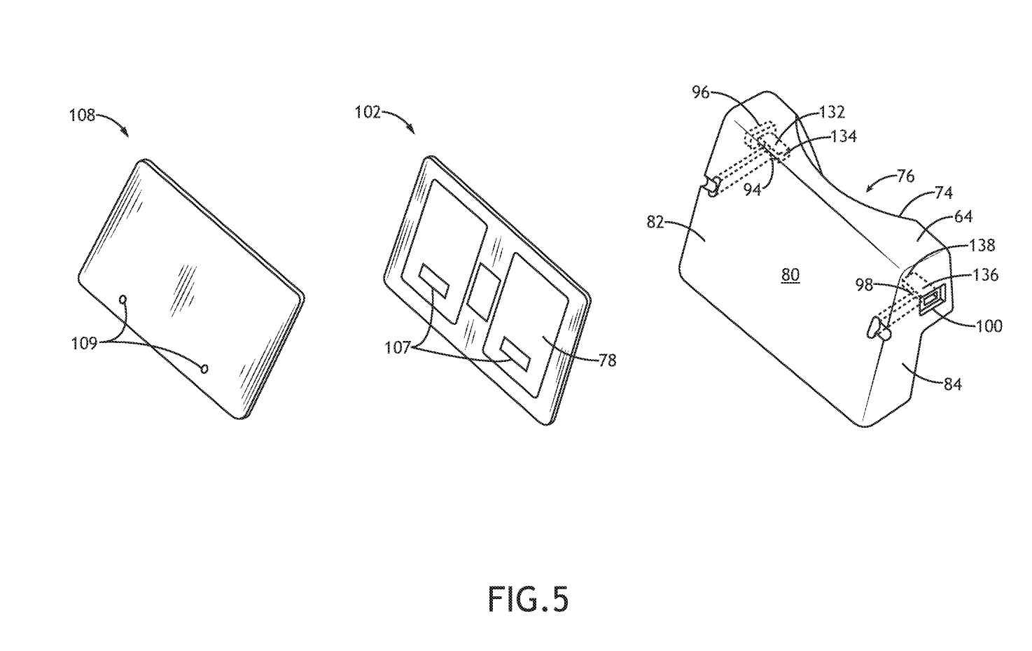

A contoured rigid plate 102, as seen in FIG. 5, is made of acrylonitrile butadiene styrene plastic is positioned in bellow 77 and is embedded in the cushion 76. Plate 102 has contoured receptors 78 to house the bladders of bladder system 110. The pump 186 is secured to plate 102, holding it permanently in place. Plate 102 also houses notches 107. The numeral 108 refers to a stiffener plate, also made of acrylonitrile butadiene styrene plastic, which is enclosed in the outer panel 54 to add rigidity. The stiffener plate 108 attaches the bladder system 110 to the outer panel 54 through notches 109 in the stiffener plate 108. The bladder system 110 is positioned in the bellow 77 between the contoured rigid plate 102 and stiffener plate 108 the air hose system 120 pass through notches 107.

An air hose system 120, as seen in FIG. 7, extends through cushion 76, through passageway 94 in cushion 76 and into an opening 96 in side 82 of cushion 76. The air hose system 120 also extends through cushion 76, through passageway 98 and into opening 100 in side 84 of cushion 76. Deflation valve 132 is positioned in opening 96 in side 82 of cushion 76 and deflation valve opening 134 found in the upstanding first side panel 28 of case 22. Deflation valve 136 is positioned in opening 100 in side 84 of cushion 76 and deflation valve opening 138 found in second side panel 30 of the case 22, as seen in FIG. 5, so as to be in communication with the air hose system 120, as seen in FIG. 7.

The bladder system 110, upon being moved between its collapsed position and the inflated position, causes the outer panel 54 to be moved from its first position towards its second position of abduction. Positions of abduction include 20°, 45°, 60°, 75°, 90° and beyond. A pump 186 allows for continuous variable timed therapy sessions customized to the patient and the rehabilitation process.

The pump, 186 located inside the bellow 77, is operated by a hand-held controller 190 to inflate and deflate the bladder system 110, as seen in FIG. 7. The hand-held controller 190 is attached to the pump 186 through a hand-held controller cord 195 which runs through the port 196. The hand-held controller 190 houses a power button 205, push buttons for degrees of abduction 206, and push buttons for therapy time intervals 207. The bladder system 110 automatically deflates once the therapy time interval is complete. When not in use the hand-held controller 190 is housed in one of two fabric controller pouches 191 which are attached to the first and second side panels 28 and 30.

A power cord 194 connects to the pump 186 and travels through the port 196. The cord 194 houses a female receptor 192 which connects to the male power cord connector 197 of the power cord 200 which connects to a source of power, as seen in FIG. 9. Although the preferred embodiment depicted in FIG. 9 includes a power cord which connects to an electric outlet, those in the art will understand that the power source can comprise a battery also functioning as a power source.

Circular clips 156 and 160 are secured to the side panels 28 and 30 of case 22 respectively and are used to connect a length adjustable shoulder strap 164 to the case 22 as seen in FIG. 3. An elongated cushion member or pad 166 is located on the length adjustable shoulder strap 164. Alternative fasteners are available that would be suitable for alternative embodiments of circular clips. Examples are D-rings, velcro, delrin clip and buckle, plastic side release buckles or cam lock buckles or any other similar clips with buckles. Those in the art will understand that in any suitable clip, now known or hereafter developed, may be used in forming the shoulder and arm support described herein.

A removable support member 198, as seen in FIG. 6, houses a removable support member cushion 199 attaches to the case 22 by a strip of hook 81 of a hook-and-loop fastener system located on removable support member 198. The removable support member 198 consists of a horizontal bottom panel 208, a curved inner panel 209, a horizontal top panel 210, a vertical first side 211, a vertical second side 212, a vertical outer panel 213, a first inner side panel 214, and a second inner side panel 215.

D-ring connectors 158 and 162, as seen in FIG. 6, are secured to the removable support member 198 first inner side panel 214, and a second inner side panel 215. The length adjustable waist strap 170 houses a buckle 204 which adjustably secures the length adjustable waist strap 170 to the patient.

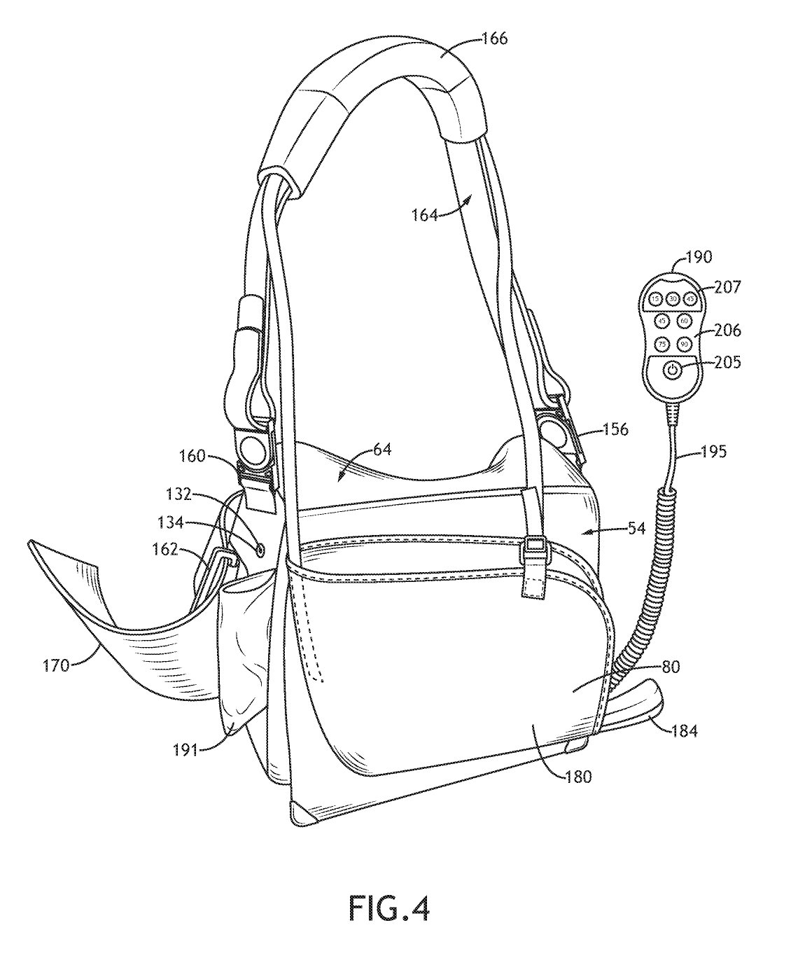

A sling 180, as seen in FIG. 4, includes an arm and elbow pouch 181 in the middle of two lengths of adjustable sling strap 183 joined together by a D-ring 185 houses a thumb loop 182 and a detachable hand support 184 used to prevent sling migration and keep the hand in neutral position. The sling which is made of fuzzy, loop or fleece fabric 80 is attached to the case 22 by a strip of hook 81 of the hook-and-loop fastener system housed on the outer panel 54, as seen in FIG. 6.

Split alignment brand marks 202, as seen in FIG. 10, are located on the upstanding first side panel 34 and upstanding second side panel 35 of the upstanding curved inner panel 74 and the first inner side panel 211 and second inner side panel 212 of the removable support member 198 to accommodate either a left arm and shoulder or a right arm and shoulder.

The advantages of the present invention are numerous. First, the instant invention can create continuous passive motion of the shoulder from 20 degrees of abduction to over 90 degrees of abduction thereby decreasing the risk of adhesive capsulitis and to promote better physiological healing. The device of this invention can also be used in a fixed position for comfort or protection of a surgical repair that is better suited by a customized, static position.

Thus, it can be seen that the invention accomplishes at least all of its stated objectives. Although the invention has been described in language that is specific to certain structures and methodological steps, it is to be understood that the invention defined in the appended claims is not necessarily limited to the specific structures and/or steps described. Rather, the specific aspects and steps are described as forms of implementing the claimed invention. Since many embodiments of the invention can be practiced without departing from the spirit and scope of the invention, the invention resides in the claims hereinafter appended.

1. A device for supporting and treating a patient's shoulder and arm, comprising:
a. a case configured to be secured to the side of the patient, wherein the said case houses a top panel, an inner panel, a bottom upstanding inner panel, a flat vertical back panel, a bottom panel, a first side panel, a second side panel, wherein each defines an interior compartment;
b. an outer panel having and an upper end, a lower end, a first side, and a second side, wherein the said upper end of the said outer panel is pivotally secured to the outer end of the said top panel so as to be movable between a first and second position of abduction;
c. a flexible bellow interconnected between the said outer panel and the said case;
d. a resilient cushion, positioned in the said interior compartment of the said case;
e. a bladder system which allow an air hose system to inflate or deflate the said bladder system, causing the said outer panel to move between the said first and second positions of abduction;
f. a contoured rigid plate which is positioned between the said resilient foam cushion and the said bladder system;
g. a stiffener plate, located in the said outer panel which is pivotally secured to the said top panel,
h. a pump located inside the said bellow which connects to the said bladder system through the said air hose system wherein the said air hose system pass through a T-connector and extend outward through the said cushion;
i. deflation valves connect to the said air hose system and the said bladder system;
j. a power cord runs from the said pump through a port located on the said bottom panel of the said case and connects to a power source;
k. a hand-held controller cord runs from the said pump through the said port located on the said bottom panel of the said case and connects to a hand-held controller;
l. said hand-held controller houses a power button, push buttons for customized degrees of abduction and push buttons for customized therapy times;
m. a controller pouch is used to house the said hand-held controller when not in use and is attached to the said first and second side panels of the said case;
n. a removable support member including an inner panel, an outer panel, a first side panel, a second side panel, an upper panel and a bottom panel creating an interior compartment is attached to the said case through a hook-and-loop fastener system;
o. a resilient foam cushion is positioned in the said interior compartment of the said removable support member;
p. a length adjustable waist strap is attached to the said removable support member by d-rings and stitching or the like;
q. a buckle is housed on the said adjustable waist strap allowing the shoulder and arm support to be secured to the patient;
r. a sling, which houses a pouch, sling strap, elongated shoulder cushion, thumb loop, detachable hand support, and d-rings, attaches to the said removable support member through a hook-and-loop fastener system;
s. a plurality of length adjustable arm straps adjustably are secured to the said exterior surface of the said outer panel;
t. a length adjustable shoulder strap attaches to the said case through circular clips located on the said first and second side panels and houses an elongated shoulder cushion;
u. alignment brand marks are located on the said case and the said removable support member to assist with assembly.

2. A device for supporting and treating a patient's shoulder and arm including removable components that can be used simultaneously or separately throughout the rehabilitation process with the combinations comprising:
a. said case, said removable support member, and a sling;
b. said removable support member and said sling;
c. said sling only.

3. A device for supporting and treating a patient's shoulder and arm, comprising:
a. a case configured to be secured to the side of the patient wherein said case defines an interior compartment;
b. an outer panel pivotally secured to the said case to be movable between a first and second position of abduction;
c. a resilient cushion, positioned in the said interior compartment of the said case;
d. a flexible bellow interconnecting between the said outer panel and said case;
e. a bladder system which houses an air hose system that allows the bladders to be inflated or deflated causing the said outer panel to move between the said first and second positions wherein the said positions of abduction;
f. said outer panel is able to be moved continuously from the said first and second positions of abduction during a timed therapy session powered by a pump which inflates and deflates the said bladder system;
g. a contoured rigid plate, which is positioned between the said resilient foam cushion and the said bladder system,
h. a stiffener plate, located in the said outer panel is pivotally secured to the said outer panel;
i. a pump located inside the said bellow connects to the said bladder system through the said air hose system;
j. deflation valves located on the said first and second side panels connect to the said air hose system and the said bladder system;
k. a hand-held controller cord runs from the said pump through the said port located on the bottom panel of the said case and connects to a hand-held controller;
l. said hand-held controller houses push buttons that activate the said pump;
m. a controller pouch used to house the said hand-held controller when not in use is attached to the said first and second side panels of the said case;
n. a power cord runs from the said pump through a port located on the said bottom panel of the said case and connects to a power source;
o. a removable support member is configured to be secured to the side of the patient wherein the said removable support member defines an interior compartment and is attached to the said case through a hook-and-loop fastener system;
p. a resilient foam cushion is positioned in the said interior compartment of the said removable support member;
q. a length adjustable waist strap is attached to the said removable support member by d-rings and stitching or the like;
r. a buckle is housed on the said waist strap allowing the shoulder and arm support to be secured to the patient;
s. a sling attaches to the said removable support member through a hook-and-loop fastener system;
t. a plurality of length adjustable arm straps are adjustably secured to the said exterior surface of the said outer panel
u. a length adjustable shoulder strap attaches to the said case and houses an elongated shoulder cushion;
v. alignment brand marks are located on the said case and the said removable support member to assist with assembly.

4. A device for supporting and treating a patient's shoulder and arm, comprising:
a. a case which houses a cushion and conforms to the patient;
b. an outer panel which connects to the said case and houses a stiffener plate and pivotally moves from the first position of abduction to the second position of abduction with respect to vertical;
c. a removable support member which comprises a cushion that conforms to the patient and attaches to the said case;
d. a flexible bellow interconnected between the said outer panel and the said case;
e. a bladder system, comprising of hoses and multiple bladders, that inflate, deflate and move the said outer panel from the first position of abduction to the second position of abduction, repeatedly, located inside the said bellow;
f. an electric pump that uses air to inflate and deflate the said bladder system, located inside the said bellow;
g. a hand-held controller is connected to the pump and houses push buttons that engage the pump;
h. straps are attached to the said case and allow the said case to be secured and customized to the wearer.

5. The device of claim 1 wherein said case may be positioned at the left side of the patient or the right side of the patient.

6. The device of claim 1 wherein the said case, the said removable support member, straps, and sling are able to fit most-to-all sizes, making it universal.

7. The device of claim 1 wherein the said alignment brand marks are used to properly align the case with the removable support member.

8. The shoulder and arm support of claim 1 wherein the said bladder system contains one or more bladders that pneumatically moves between a collapsed position and an inflated position.

9. The device of claim 1 wherein the said pump automatically powers off and the said bladder system automatically deflates once the therapy time interval is complete.

10. The device of claim 1 wherein the said sling is made of mesh, fleece or loop fabric.

11. The device of claim 1 wherein the said hand-held controller pouch is made of fleece or loop fabric.

12. The device of claim 1 wherein the said case is made of fleece or loop fabric.

13. The device of claim 1 wherein the said resilient cushion is made of polyurethane foam.

14. The device of claim 1 wherein the hand-held controller is made of acrylonitrile butadiene styrene plastic.

15. The device of claim 1 wherein the said bladder is made of thermoplastic polyurethane.

16. The device of claim 1 wherein the said bellow is made of polyester.

17. The device of claim 1 wherein the said contoured rigid plate and the said stiffener plate are made of acrylonitrile butadiene styrene plastic.

18. The device of claim 1 wherein the said detachable hand support can be attached to the said case, the said removable support member or the said sling.
